(19) United States
(12) Patent Application Publication
(10) Pub. No.: US 2026/0060702 A1
(43) Pub. Date: Mar. 5, 2026

(54) MEDICAL DEVICE, SYSTEM AND ASSOCIATED METHODS

(71) Applicant: ASAHI INTECC CO., LTD., Seto-shi (JP)

(72) Inventors: Makoto NISHIGISHI, Seto-shi (JP); Hirotomo SHIMIZU, Seto-shi (JP); Yoshiki KATO, Seto-shi (JP); Yusuke KIDO, Seto-shi (JP)

(73) Assignee: ASAHI INTECC CO., LTD., Seto-shi (JP)

(21) Appl. No.: 19/382,284

(22) Filed: Nov. 7, 2025

Related U.S. Application Data
(63) Continuation of application No. PCT/JP2024/005453, filed on Feb. 16, 2024.

(30) Foreign Application Priority Data
May 19, 2023 (JP) 2023-083071

(51) Int. Cl. A61B 17/22; A61B 17/00; A61B 90/00

(52) U.S. Cl. CPC A61B 17/22; A61B 17/00234; A61B 90/361; A61B 2017/00238; A61B 2017/003; A61B 2017/22094

(57) ABSTRACT

This medical device comprises: a first tube having a first lumen and a second tube having a second lumen. An outer peripheral surface of the first tube and an outer peripheral surface of the second tube are joined, and in a transverse cross-section at a predetermined position in a longitudinal direction of the medical device, a first area enclosed by an outer periphery of the medical device is less than or equal to a second area enclosed by both a single common external tangent line of the first tube and the second tube, and a contour line formed by an outer periphery of the first tube and the second tube.

### CROSS-REFERENCE TO RELATED APPLICATIONS

The present application is a bypass continuation of International Application No. PCT/JP2024/005453, filed Feb. 16, 2024, which claims priority to Japanese patent application 2023-083071, filed May 19, 2023, the entire contents of each of which being incorporated herein by reference.

### TECHNICAL FIELD

The present disclosure relates to a medical device.

### BACKGROUND ART

Because procedures for opening a CTO are complex, the procedures are sometimes performed under the guidance of a sensor that acquires image information of biological tissue using ultrasound waves. In such cases, a catheter having two or more lumens is used, and by inserting a sensor through one lumen, and a therapeutic device through another lumen, the sensor and the therapeutic device can be used together during the procedure. In order to improve safety and operability, such catheters generally have a structure that is thin and flexible on the distal end side, and thick and highly rigid on the proximal end side. For example, Patent Literature 1 to 3 disclose devices that can be used in such procedures. Patent Literature 4 discloses a suction catheter having a guidewire lumen and a suction lumen. Patent Literature 5 discloses an irrigated catheter that utilizes a multi-lumen irrigation tubing to achieve irrigation flow.

### CITATION LIST

### Patent Literature

Patent Literature 1: JP 2018-033507 A

Patent Literature 2: JP 2015-180275 A

Patent Literature 3: JP 2013-34652 A

Patent Literature 4: JP 2010-57831 A

Patent Literature 5: JP 2016-5641 A

### SUMMARY

### Technical Problems

Here, in a PCI procedure, in addition to the catheters described above, a guiding catheter is sometimes used to collectively guide a plurality of devices used in the procedure, such as a guidewire or a microcatheter to the site of the CTO lesion.

Hereinafter, additional devices that are inserted through the guiding catheter are referred to collectively as “auxiliary devices”. Although a guiding catheter has a lumen large enough to accommodate a plurality of devices, the size of the lumen is restricted because the guiding catheter is inserted into a blood vessel. Accordingly, medical devices have been sought that enable the use of as many auxiliary devices as possible inside the limited lumen of the guiding catheter. However, the devices described in Patent Literature 1 to 5 do not address this issue in any way.

Note that, such a problem is not limited to PCI procedures such as opening a CTO, and is generally shared by medical devices used in percutaneous procedures.

Furthermore, such a problem is not limited to the vascular system, and is common to medical devices inserted into various organs within the human body, such the lymphatic system, biliary system, urinary tract system, respiratory system, digestive system, secretory glands, and reproductive organs.

The present disclosure has been made to solve at least part of the problem described above, and is directed to allowing, in a medical device having two or more lumens, the use of many auxiliary devices inside the limited lumen of a guiding catheter.

### Solutions to Problems

The present disclosure has been made to solve at least part of the problem described above and others, and can be realized as the following aspects.

According to an aspect of the present disclosure, a medical device is provided. The medical device includes: a first tube having a first lumen; a second tube having a second lumen; and a third tube having a third lumen; wherein an outer peripheral surface of the first tube and an outer peripheral surface of the second tube are joined, and in a transverse cross-section at a predetermined position in a longitudinal direction of the medical device, a first area, being an area of a region that is enclosed by an outer periphery of the medical device, is less than or equal to a second area, being an area of a region that is enclosed by both a single common external tangent line of the first tube and the second tube, and a contour line formed by an outer periphery of the first tube and the second tube.

Note that the present disclosure can be implemented in various forms, such as in the form of a medical device, a catheter, manufacturing methods of the same, and the like.

### BRIEF DESCRIPTION OF DRAWINGS

FIG. 1 is an explanatory diagram illustrating a configuration of a catheter serving as a medical device.

FIG. 2 is an explanatory diagram illustrating a configuration of a catheter serving as a medical device.

FIGS. 3A to 3F are transverse cross-sectional views of the catheter.

FIG. 4 is a diagram describing a method of using the catheter.

FIG. 5 is a diagram describing a method of using the catheter.

FIGS. 6A and 6B are diagrams describing a common external tangent line.

FIG. 7 is a diagram describing a first area and a second area.

FIG. 8 is a diagram describing a first area and a third area.

FIGS. 9A and 9B are diagrams describing the insertion of a guiding catheter.

FIG. 10 is a transverse cross-sectional view of a catheter according to a second embodiment.

FIGS. 11A and 11B are transverse cross-sectional views of a catheter according to a third embodiment.

### DETAILED DESCRIPTION

### First Embodiment

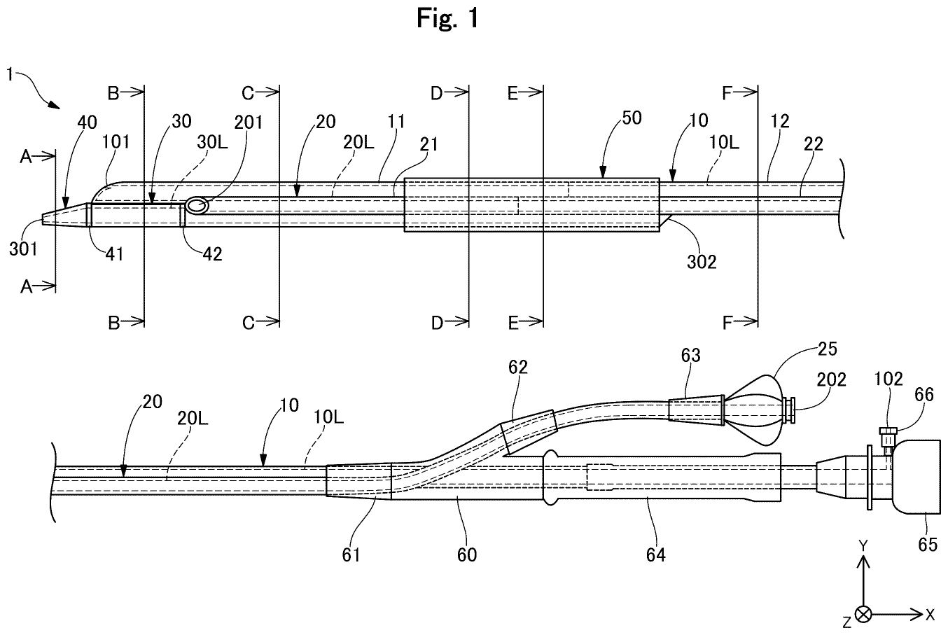

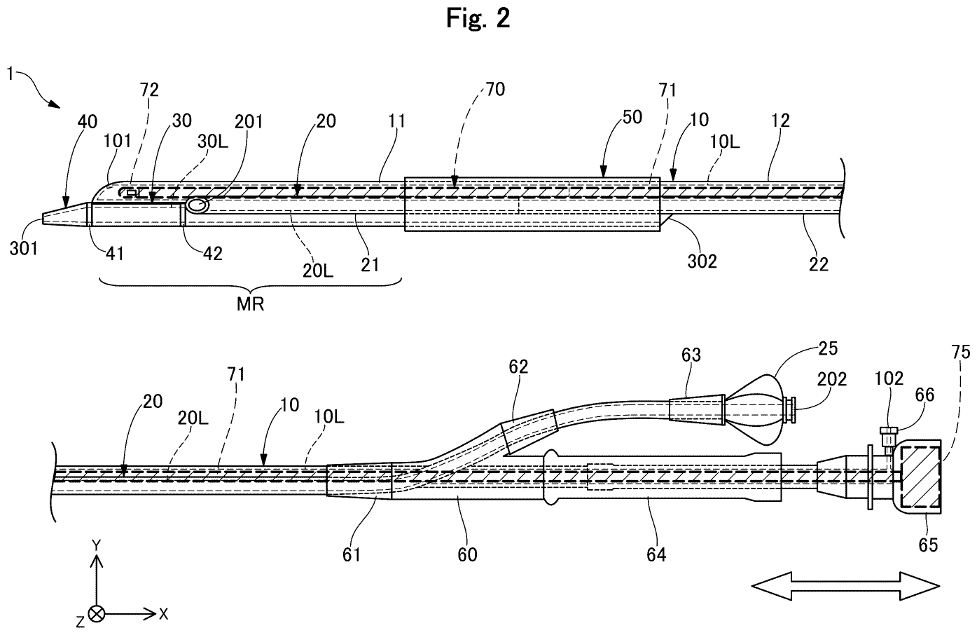

FIGS. 1 and 2 are explanatory diagrams illustrating the configuration of a medical device 1. The medical device 1 according to the present embodiment is a catheter used for treating a lesion in a biological lumen, such as a CTO that has occurred in a blood vessel. Hereinafter, the medical device 1 is also referred to as “catheter 1”. As shown in FIGS. 1 and 2, the catheter 1 includes a sensor tube 10, an OTW (Over The Wire) tube 20, an RX (Rapid Exchange) tube 30, a distal end tip 40, a first marker 41, a second marker 42, a covering portion 50, a branched connector 60, a first reinforcing member 61 to a third reinforcing member 63, a cylindrical member 64, a connector 65, a connector 25, and a sensor 70.

In FIG. 1, illustration of the sensor 70 is omitted in order to describe the configuration of the tube and the lumen inside the tube. In FIG. 2, the sensor 70, which is built into a sensor lumen 10L inside the sensor tube 10, is illustrated by a dashed line with diagonal hatching.

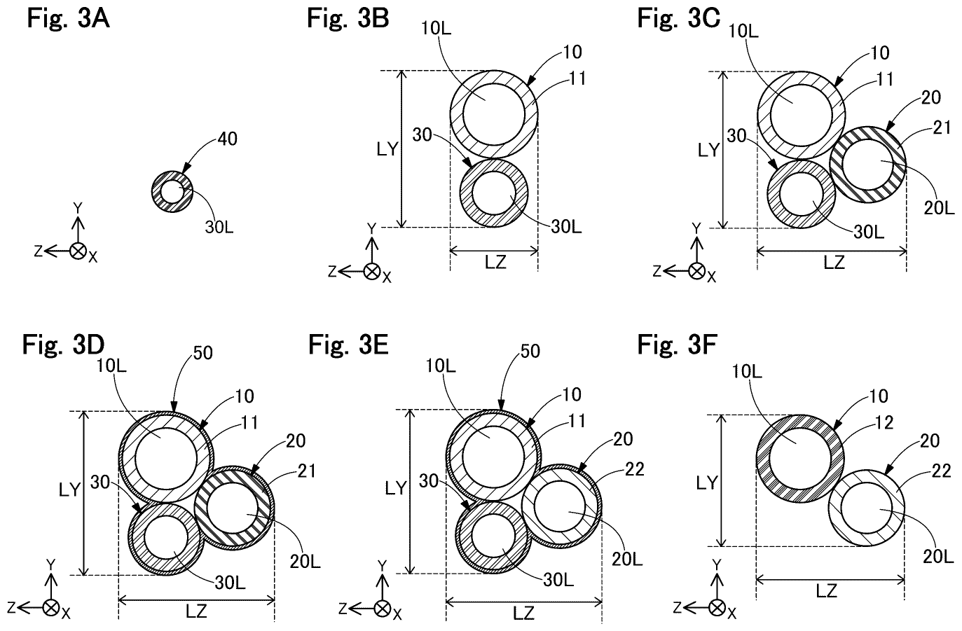

For convenience of the description, FIGS. 1 and 2 include parts where the components are illustrated with a relative size ratio which is different from the actual relative size ratio. Also, portions in which the components are shown exaggerated may be included. Furthermore, FIGS. 1 and 2 show mutually orthogonal XYZ axes. The X-axis corresponds to the longitudinal direction of the catheter 1, the Y-axis corresponds to the height direction of the catheter 1, and the Z-axis corresponds to the width direction of the catheter 1. The left side (negative X-axis direction) in FIGS. 1 and 2 is referred to as the “distal end side” of the catheter 1 and the components thereof, and the right side (positive X-axis direction) in FIGS. 1 and 2 is referred to as the “proximal end side” of the catheter 1 and the components thereof. Also, of the two ends in the longitudinal direction (X-axis direction) of the catheter 1 and the components thereof, one end positioned on the distal end side is referred to as the “distal end”, and the other end positioned on the proximal end side is referred to as the “proximal end”. In addition, the distal end and the vicinity thereof is referred to as a “distal end portion”, and the proximal end and the vicinity thereof is referred to as the “proximal end portion”. The distal end side is inserted into the living body, and the proximal end side is operated by an operator, such as a physician. These points are also common to FIGS. 3A to 3F and subsequent drawings.

FIGS. 3A to 3F are transverse cross-sectional views of the catheter. FIG. 3A shows a transverse cross-section of the catheter 1 along line A-A in FIG. 1. FIG. 3B shows a transverse cross-section of the catheter 1 along line B-B in FIG. 1. FIG. 3C shows a transverse cross-section of the catheter 1 along line C-C in FIG. 1. FIG. 3D shows a transverse cross-section of the catheter 1 along line D-D in FIG. 1. FIG. 3E shows a transverse cross-section of the catheter 1 along line E-E in FIG. 1. FIG. 3F shows a transverse cross-section of the catheter 1 along line F-F in FIG. 1. Hereinafter, the configuration of the catheter 1 will be described using FIGS. 1, 2, and 3A to 3F.

The sensor tube 10 is a cylindrically-shaped member (tubular body) having an elongated outer shape. The sensor tube 10 extends linearly along the longitudinal direction (X-axis direction) of the catheter 1, parallel to the OTW tube 20 and the RX tube 30. Inside the sensor tube 10, the sensor lumen 10L (dashed line) for accommodating the sensor 70 is formed. The sensor lumen 10L is a lumen for the sensor 70.

The distal end of the sensor tube 10 is positioned at the same position as the distal end of the RX tube 30 in the longitudinal direction of the catheter 1, or a position slightly toward the proximal end side. A distal end opening 101 is formed in the distal end of the sensor tube 10, communicating the distal end of the sensor lumen 10L with the outside. The distal end opening 101 is a fluid discharge port for keeping the inside of the sensor lumen 10L in a wet state. The proximal end of the sensor tube 10 is positioned further toward the proximal end side than the proximal end of the OTW lumen 20L and the proximal end of the RX tube 30 in the longitudinal direction of the catheter 1. On the proximal end side of the sensor tube 10, the first reinforcing member 61, the branched connector 60, the cylindrical member 64, and the connector 65 are attached in this order from the distal end side toward the proximal end side. The details will be described later. A fluid supply portion 66 is attached to the connector 65, and a proximal end opening 102 is formed in the fluid supply portion 66, communicating the proximal end of the sensor lumen 10L with the outside. The proximal end opening 102 is a fluid supply port to the sensor lumen 10L.

As shown in FIG. 1, the sensor tube 10 has a distal end side tube 11 that is disposed on the distal end side, and a proximal end side tube 12 that is disposed further toward the proximal end side than the distal end side tube 11. The distal end side tube 11 and the proximal end side tube 12 are both cylindrical members (tubular bodies) having an elongated outer shape. The distal end side tube 11 and the proximal end side tube 12 are connected at an arbitrary location where the covering portion 50 is provided in the longitudinal direction. That is, the distal end side tube 11 and the proximal end side tube 12 each constitute a portion of the sensor lumen 10L.

The OTW tube 20 is a cylindrically-shaped member (tubular body) having an elongated outer shape. On the distal end side of the branched connector 60, the OTW tube 20 extends linearly along the longitudinal direction of the catheter 1, parallel to the sensor tube 10 and the RX tube 30. Inside the OTW tube 20, an OTW lumen 20L (dashed line) for accommodating a therapeutic device (for example, a plasma guidewire or a penetration guidewire) is formed. The OTW lumen 20L is a so-called over-the-wire (OTW) type lumen that does not have a proximal end opening in the part placed inside the biological lumen during use of the catheter 1.

The distal end of the OTW tube 20 is positioned further toward the proximal end side than the distal end of the sensor tube 10 and the distal end of the RX tube 30 in the longitudinal direction of the catheter 1. A distal end opening 201 is formed in the distal end of the OTW tube 20, communicating the distal end of the OTW lumen 20L with the outside. The distal end opening 201 is a device protrusion port for causing the therapeutic device to protrude toward biological tissue. As a result of the distal end portion of the OTW tube 20 being diagonally cut, the distal end opening 201 faces a direction intersecting the longitudinal direction of the catheter 1. As a result, during use of the catheter 1, it becomes easier to make the therapeutic device reach the biological tissue that is present around the catheter 1. The proximal end of the OTW tube 20 is positioned further toward the distal end side than the proximal end of the sensor tube 10, and further toward the proximal end side than the proximal end of the RX tube 30 in the longitudinal direction of the catheter 1. On the proximal end side of the OTW tube 20, the first reinforcing member 61, the branched connector 60, the second reinforcing member 62, the third reinforcing member 63, and the connector 25 are attached in this order from the distal end side toward the proximal end side. The details will be described later. A proximal end opening 202 is formed in the connector 25, communicating the proximal end of the OTW lumen 20L with the outside. The proximal end opening 202 is a device insertion port for inserting the therapeutic device into the OTW lumen 20L.

As shown in FIG. 1, the OTW tube 20 has a distal end side tube 21 that is disposed on the distal end side, and a proximal end side tube 22 that is disposed further toward the proximal end side than the distal end side tube 21. The distal end side tube 21 and the proximal end side tube 22 are both cylindrical members (tubular bodies) having an elongated outer shape. The distal end side tube 21 and the proximal end side tube 22 are connected at an arbitrary location where the covering portion 50 is provided in the longitudinal direction. That is, the distal end side tube 21 and the proximal end side tube 22 each constitute a portion of the OTW lumen 20L.

The RX tube 30 is a cylindrically-shaped member (tubular body) having an elongated outer shape. The RX tube 30 extends linearly along the longitudinal direction of the catheter 1, parallel to the sensor tube 10 and the OTW tube 20. Inside the RX tube 30, an RX lumen 30L (dashed line) for accommodating a workhorse wire is formed.

The distal end of the RX tube 30 is positioned at the same position as the distal end of the sensor tube 10 in the longitudinal direction of the catheter 1, or a position slightly toward the distal end side. A hollow distal end tip 40 is joined to the distal end portion of the RX tube 30. A distal end opening 301 is formed in the distal end of the distal end tip 40, communicating the distal end of the RX lumen 30L with the outside. The distal end opening 301 is a wire insertion port for inserting the workhorse wire into the RX lumen 30L. The proximal end of the RX tube 30 is positioned further toward the distal end side than the proximal end of the sensor tube 10 and the proximal end of the OTW tube 20 in the longitudinal direction of the catheter 1. A proximal end opening 302 is formed in the proximal end of the RX tube 30, communicating the proximal end of the RX lumen 30L with the outside. The proximal end opening 302 is a wire withdrawal port for withdrawing the workhorse wire to the outside. As a result of the proximal end of the RX tube 30 being diagonally cut, the proximal end opening 302 faces a direction intersecting the longitudinal direction of the catheter 1. As a result, during use of the catheter 1, it becomes easier to withdraw the workhorse wire from the proximal end opening 302.

The distal end tip 40 is a cylindrical member that is radiopaque and has an outer diameter that increases from the distal end side toward the proximal end side. As a result of being joined to the distal end portion of the RX tube 30, the distal end tip 40 is positioned at the distal end of the catheter 1, and advances through a biological lumen ahead of the other members. The inner cavity of the distal end tip 40 communicates with the RX lumen 30L of the RX tube 30, and as described above, the distal end opening 301, which communicates the distal end of the RX lumen 30L with the outside, is formed in the distal end of the distal end tip 40.

The first marker 41 and the second marker 42 are annular members having radiopacity. The first marker 41 is disposed at a position adjacent to the proximal end of the distal end tip 40 on the outer peripheral surface of the RX tube 30, and is joined to the outer peripheral surface of the RX tube 30. The second marker 42 is disposed at a position adjacent to the distal end of the distal end opening 201 of the OTW tube 20 on the outer peripheral surface of the RX tube 30, and is joined to the outer peripheral surface of the RX tube 30. The joining of the first marker 41 and the second marker 42, for example, can be performed by joining resins to each other by heat fusion, or by joining using an adhesive such as an epoxy-based adhesive. The first marker 41 and the second marker 42 may be colored such that the markers can be directly observed by an operator. In this way, by disposing the first marker 41 and the second marker 42 on the RX tube 30, it is possible to suppress interference in the sensing (acquisition of image information) by the sensor 70 caused by the first marker 41 or the second marker 42.

As shown in FIG. 3A, only the distal end tip 40 is present in the transverse cross-section along line A-A. As shown in FIG. 3B, in the transverse cross-section along line B-B, the sensor tube 10 (specifically, the distal end side tube 11) and the RX tube 30 have the outer peripheral surfaces joined to each other. As shown in FIG. 3C, in the transverse cross-section along line C-C, the sensor tube 10 (specifically, the distal end side tube 11), the OTW tube 20 (specifically, the distal end side tube 21), and the RX tube 30 have the outer peripheral surfaces joined to each other. As shown in FIG. 3D, in the transverse cross-section along line D-D, the sensor tube 10 (specifically, the distal end side tube 11), the OTW tube 20 (specifically, the distal end side tube 21), and the RX tube 30 have the outer peripheral surfaces joined to each other. Furthermore, in the D-D transverse cross-section, the covering portion 50 fixes the three tubes 10, 20, and 30 by covering the part of the surface of the three tubes 10, 20, and 30 facing the outer side along the outer periphery of the three tubes 10, 20, and 30. As shown in FIG. 3E, in the transverse cross-section along line E-E, the sensor tube 10 (specifically, the distal end side tube 11), the OTW tube 20 (specifically, the proximal end side tube 22), and the RX tube 30 have the outer peripheral surfaces joined to each other. Similarly, in the E-E transverse cross-section, the covering portion 50 fixes the three tubes 10, 20, and 30 by covering the part of the surface of the three tubes 10, 20, and 30 facing the outer side along the outer periphery of the three tubes 10, 20, and 30. As shown in FIG. 3F, in the transverse cross-section along line F-F, the sensor tube 10 (specifically, the proximal end side tube 12) and the OTW tube 20 (specifically, the proximal end side tube 22) have the outer peripheral surfaces joined to each other.

The joining of the sensor tube 10, the OTW tube 20, and the RX tube 30 may be performed using an arbitrary joining agent such as an epoxy-based adhesive, or by thermal welding. In the B-B transverse cross-section, the C-C transverse cross-section, the D-D transverse cross-section, and the E-E transverse cross-section, the height LY of the catheter 1 is larger than the width LZ of the catheter 1. On the other hand, in the F-F transverse cross-section, the height LY of the catheter 1 is smaller than the width LZ of the catheter 1. Note that, as shown in FIGS. 3A to 3F, the magnitude relationship between the outer diameters of the three tubes 10, 20, and 30 is as follows: outer diameter of sensor tube 10>outer diameter of OTW tube 20>outer diameter of RX tube 30. Furthermore, the magnitude relationship between the inner diameters (lumens) of the three tubes 10, 20, and 30 is as follows: inner diameter of sensor lumen 10L>inner diameter of OTW lumen 20L>inner diameter of RX lumen 30L. However, these magnitude relationships of the outer diameters and inner diameters are merely an example, and may be arbitrarily changed.

The description will be continued returning to FIG. 1. The covering portion 50 is a resin layer for fixing the three tubes 10, 20, and 30 (specifically, the sensor tube 10, the OTW tube 20, and the RX tube 30). The covering portion 50 is provided in a section where the three tubes 10, 20, and 30 extend side-by-side, or in other words, in a section further toward the proximal end side than the distal end opening 201, and further toward the distal end side than the proximal end opening 302. Note that, as shown in FIG. 1, the covering portion 50 may be provided further toward the proximal end side than the distal end opening 201, or in other words, at a position separated from the distal end opening 201 toward the proximal end side. This can suppress interference in the sensing (acquisition of image information) by the sensor 70, which has been inserted in the sensor lumen 10L, caused by the covering portion 50. As shown in FIGS. 3D and 3E, the covering portion 50 thinly covers the parts of the three tubes 10, 20, and 30 facing the outer side along the outer periphery of the three tubes 10, 20, and 30. As a result, the covering portion 50 is capable of fixing the three tubes 10, 20, and 30 while maintaining the recesses (concave portions) formed at the parts adjacent to each of the tubes 10, 20, and 30 in the transverse cross-section of the catheter 1.

The branched connector 60 is a member having an inner cavity that branches into two branches, and is disposed on the proximal end side of the catheter 1. The OTW tube 20 is inserted through one inner cavity of the branched connector 60. The sensor tube 10 is inserted through another inner cavity of the branched connector 60. The first reinforcing member 61 is a cylindrical member disposed further toward the distal end side than the branched connector 60. The first reinforcing member 61 covers the outer periphery of each of the sensor tube 10 and the OTW tube 20 that have been inserted into the branched connector 60, which reinforces the distal end side of the branched connector 60.

The second reinforcing member 62 is a cylindrical member disposed further toward the proximal end side of the one branch of the branched connector 60. The second reinforcing member 62 covers the outer periphery of the OTW tube 20 that has been inserted into the branched connector 60, which reinforces the proximal end side of the branched connector 60. The third reinforcing member 63 is a cylindrical member disposed further toward the distal end side than the connector 25. The third reinforcing member 63 covers the outer periphery of the OTW tube 20 that has been inserted into the connector 25, which reinforces the distal end side of the connector 25. The connector 25 is a member that is joined to the proximal end portion of the OTW tube 20. The connector 25 has a pair of wing portions for an operator to grip. The proximal end opening 202 (device insertion port) is formed at the proximal end of the connector 25, communicating the proximal end of the OTW lumen 20L with the outside.

The cylindrical member 64 is a cylindrical member disposed further toward the proximal end side of the another branch of the branched connector 60. The cylindrical member 64 covers the outer periphery of the sensor tube 10 that has been inserted into the branched connector 60, which reinforces the proximal end side of the branched connector 60. The connector 65 is a member that is joined to the proximal end portion of the sensor tube 10. A housing for accommodating a connection terminal 75 of the sensor 70 is provided on the proximal end side of the connector 65. On the outer peripheral surface of the connector 65, a fluid supply portion 66 is provided, in which a proximal end opening 102 that communicates the proximal end of the sensor lumen 10L with the outside is formed.

The sensor 70 (FIG. 2) is an imaging sensor for acquiring image information. As shown in FIG. 2, the sensor 70 includes a main body portion 71, a probe 72, and a connection terminal 75. The main body portion 71 is an elongated member that extends along the longitudinal direction of the catheter 1. A driving cable (coaxial cable) that electrically connects the probe 72 and the connection terminal 75 is built into the inside of the main body portion 71. The probe 72 includes an ultrasonic transducer (also referred to as an ultrasonic vibrator, a piezoelectric body, an ultrasonic transmission/reception element, or an ultrasonic element) that transmits ultrasonic waves toward biological tissue, and receives the ultrasonic waves that have propagated through the biological tissue and have then been reflected. The probe 72 is also referred to as an imaging core or a transducer. The connection terminal 75 is a terminal that electrically connects the sensor 70 to a console terminal provided externally. The connection terminal 75 is provided at the proximal end of the main body portion 71, and is accommodated inside the housing of the connector 65.

The sensor 70 is electrically connected to an external console terminal via the connection terminal 75, receives a supply of power from the console terminal, and outputs detection signals from the probe 72 to the console terminal. As a result, the console terminal is capable of displaying image information based on the detection signals from the probe 72. As shown in FIG. 2, the sensor 70 is fixed to the connector 65. Furthermore, as indicated by the white arrows in FIG. 2, by gripping and sliding the connector 65 and in the front-rear direction (the direction of the white arrow), an operator is capable of moving the position of the probe 72 of the sensor 70 within a range MR from the distal end of the sensor lumen 10L to the distal end of the covering portion 50, or in other words, within a predetermined range MR that includes the distal end opening 201.

The distal end side tube 11 of the sensor tube 10, the distal end side tube 21 of the OTW tube 20, the RX tube 30, and the covering portion 50 can be formed of a flexible material such as a thermoplastic resin such as polyethylene resin, polypropylene resin, or polyurethane, polyvinyl chloride, ethylene-vinyl acetate copolymer, cross-linked ethylene-vinyl acetate copolymer, polyamide elastomer, polyolefin elastomer, polyurethane elastomer, silicone rubber, or latex rubber. The distal end side tube 11 of the sensor tube 10, the distal end side tube 21 of the OTW tube 20, the RX tube 30, and the covering portion 50 may be formed of the same material, or formed of different materials.

The distal end side tube 21 of the sensor tube 10 and the proximal end side tube 22 of the OTW tube 20 can be formed of a resin having high rigidity such as nylon resin, polyester resin, or PEEK resin. The distal end side tube 21 of the sensor tube 10 and the proximal end side tube 22 of the OTW tube 20 may be formed of the same material, or formed of different materials. Note that any one or more of the distal end side tube 11 and the proximal end side tube 12 of the sensor tube 10, the distal end side tube 21 and the proximal end side tube 22 of the OTW tube 20, and the RX tube 30 may have a multi-layer configuration in which tubes of different materials are layered.

The distal end tip 40, the first marker 41, and the second marker 42 can be formed of a radiopaque resin material or metal material. For example, in a case where a radiopaque resin is used, the distal end tip 40, the first marker 41, and the second marker 42 can be formed by mixing a radiopaque material such as bismuth trioxide, tungsten, or barium sulfate with a polyamide resin, a polyolefin resin, a polyester resin, a polyurethane resin, a silicon resin, a fluororesin, or the like. For example, when a radiopaque metallic material is used, the distal end tip 40, the first marker 41, and the second marker 42 can be formed of gold, platinum, tungsten, or an alloy containing these elements (such as a platinum-nickel alloy). The distal end tip 40, the first marker 41, and the second marker 42 may be formed of the same material, or formed of different materials.

The branched connector 60, the first reinforcing member 61 to the third reinforcing member 63, the cylindrical member 64, the connector 65, and the connector 25 can be formed of a known resin material. The branched connector 60, the first reinforcing member 61 to the third reinforcing member 63, the cylindrical member 64, the connector 65, and the connector 25 may be formed of the same material, or formed of different materials.

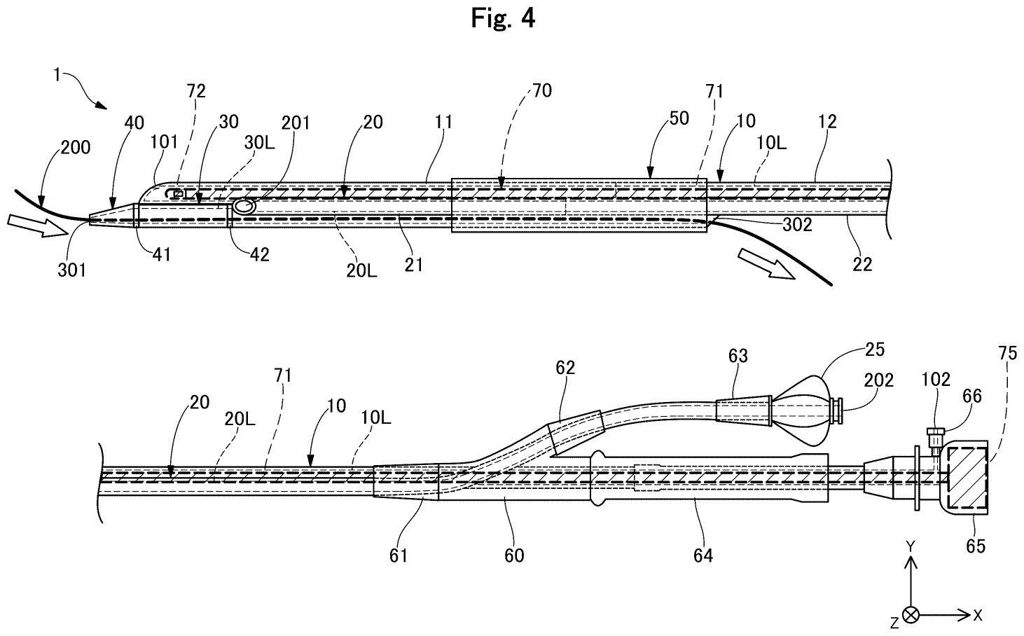

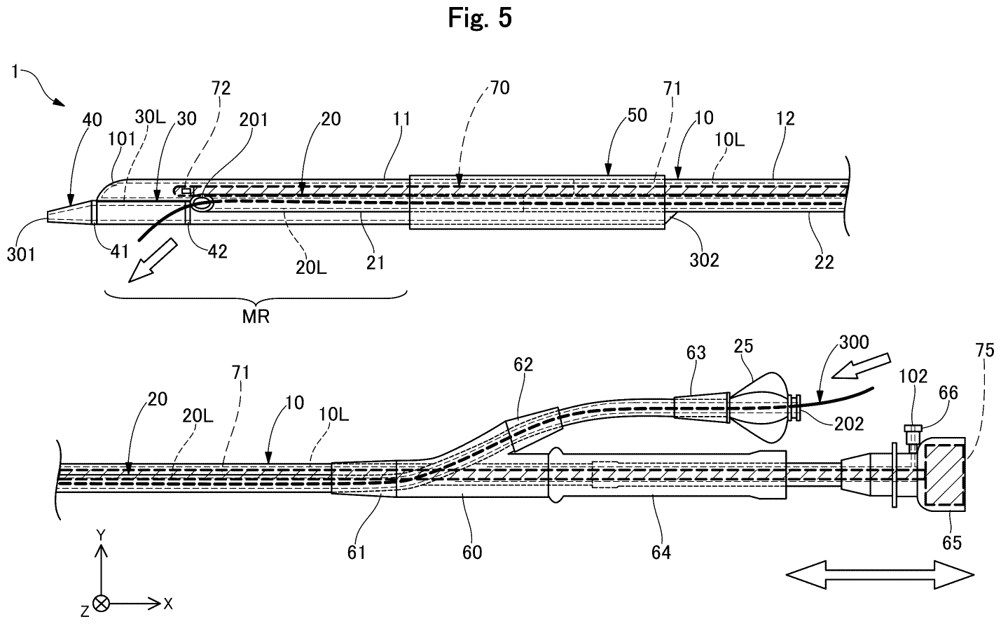

FIGS. 4 and 5 are diagrams describing a method of using the catheter 1. The following procedures a1 to a6 illustrate a case where a CTO (lesion) that has occurred in a blood vessel is recanalized using an antegrade approach. However, the catheter 1 may be used in a retrograde approach, and may be used for procedures other than CTO recanalization.

(a1) The operator inserts a workhorse wire 200 into the blood vessel, and delivers the distal end portion of the workhorse wire 200 near the CTO. (a2) The operator inserts the proximal end portion of the workhorse wire 200 from the distal end opening 301 of the catheter 1, and causes the proximal end portion of the workhorse wire 200 to pass through the RX lumen 30L, and be drawn out from the proximal end opening 302 of the catheter 1 (FIG. 4). (a3) The operator pushes the catheter 1 along the workhorse wire 200 into the blood vessel, and delivers the distal end portion of the catheter 1 near the CTO. In procedure a3, the catheter 1 may be delivered near the CTO by being passed through a guiding catheter previously inserted into the blood vessel along the workhorse wire 200. (a4) The operator grips and slides the connector 65 in the front-rear direction (FIG. 5: direction of white arrow) to adjust the position of the probe 72 of the sensor 70 within the range MR, and while checking the image displayed on the console terminal, aligns the position and orientation of the CTO and the distal end opening 201. The position refers to the position in the extending direction of the blood vessel, and the orientation refers to the orientation in the circumferential direction of the inner wall of the blood vessel. (a5) The operator inserts the distal end portion of a therapeutic device 300 from the proximal end opening 202 of the catheter 1, and causes the distal end portion of the therapeutic device 300 to be inserted through the OTW lumen 20L and protrude from the distal end opening 201 of the catheter 1 (FIG. 5). (a6) The operator treats the CTO using the therapeutic device 300 while adjusting the position of the probe 72 of the sensor 70 within the range MR as necessary, and checking the image displayed on the console terminal. As described above, an arbitrary device such as a plasma guidewire or a penetration guidewire can be used as the therapeutic device 300.

Note that the sensor tube 10, the OTW tube 20, and the RX tube 30 are also collectively referred to as a “shaft”. Furthermore, the sensor lumen 10L corresponds to a “first lumen”, and the sensor tube 10 corresponds to a “first tube”. The OTW lumen 20L corresponds to a “second lumen”, and the OTW tube 20 corresponds to a “second tube”. The RX lumen 30L corresponds to a “third lumen”, and the RX tube 30 corresponds to a “third tube”. Note that, in the present embodiment, “same” and “equal” are not limited to strictly identical cases, and allows for differences due to manufacturing error and the like. Also, “constant” has the same meaning as “substantially constant”, and means being generally constant while allowing for deviations due to manufacturing error and the like.

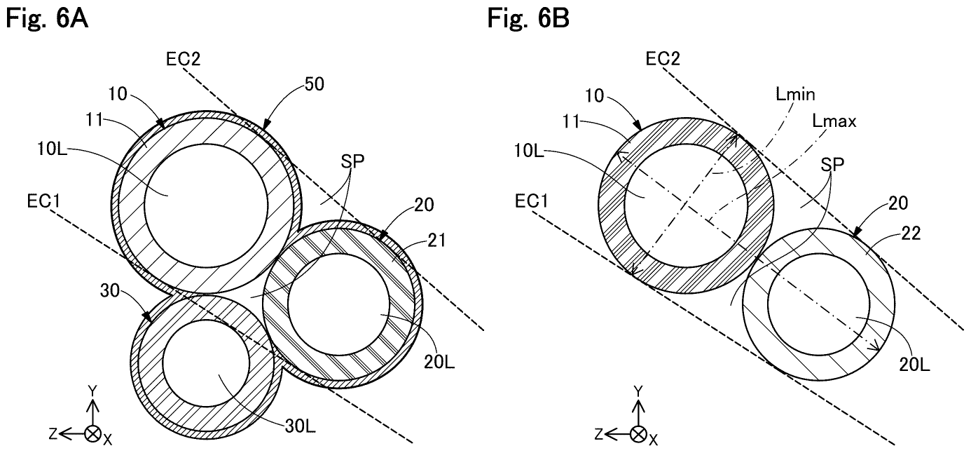

FIGS. 6A and 6B are diagrams describing a common external tangent line. FIG. 6A shows a transverse cross-section of the catheter 1 along line D-D in FIG. 1. FIG. 6B shows a transverse cross-section of the catheter 1 along line F-F in FIG. 1. In FIGS. 6A and 6B, common external tangent lines EC1 and EC2 of the sensor tube 10 (first tube) and the OTW tube 20 (second tube) are respectively indicated by dashed lines. The common external tangent line EC1 is also referred to as a “first common external tangent line EC1”, and the common external tangent line EC2 is also referred to as a “second common external tangent line EC2”.

As shown in FIG. 6A, in the transverse cross-section of the catheter 1 along line D-D, a gap SP is formed between the first common external tangent line EC1 and the outer periphery of the sensor tube 10 and the OTW tube 20. As shown in FIG. 6A, in the transverse cross-section of the catheter 1 along line D-D, a gap SP is formed between the second common external tangent line EC2 and the outer periphery of the catheter 1 (specifically, the outer periphery of the covering portion 50). In the transverse cross-section of the catheter 1 along line D-D, the first common external tangent line EC1 intersects the RX tube 30. The gap SP formed between the first common external tangent line EC1 and the outer periphery of the sensor tube 10 and the OTW tube 20 is an inner cavity formed inside the catheter 1, i.e., becomes an enclosed inner cavity of the medical device due to the presence of the covering portion 50.

As shown in FIG. 6B, in the transverse cross-section of the catheter 1 along line F-F, a gap SP is formed between the first common external tangent line EC1 and the outer periphery of the catheter 1. As shown in FIG. 6B, in the transverse cross-section of the catheter 1 along line F-F, a gap SP is formed between the first common external tangent line EC1 and the outer periphery of the sensor tube 10 and the OTW tube 20. In addition, a gap SP is formed between the second common external tangent line EC2 and the outer periphery of the catheter 1. A gap SP is formed between the second common external tangent line EC2 and the outer periphery of the sensor tube 10 and the OTW tube 20. The gaps SP correspond to “recesses (concave portions) formed at the parts adjacent to the tubes 10 and 20” mentioned above.

Further, as shown in FIG. 6B, in the transverse cross-section of the catheter 1 along line F-F, an outer diameter Lmax (maximum outer diameter) in the longitudinal direction of the catheter 1 is the outer diameter of the sensor tube 10 and the OTW tube 20, or in other words, becomes the outer diameter of the two tubes. On the other hand, an outer diameter Lmin (minimum outer diameter) in the short axis direction of the catheter 1 can be made the outer diameter of the sensor tube 10, or in other words, the outer diameter of one tube. Here, the outer diameter Lmin (minimum outer diameter) is equal to the outer diameter of the tube among the sensor tube 10 and the OTW tube 20 having the larger outer diameter.

Note that, in the longitudinal direction of the catheter 1, an arbitrary position of a part where the RX tube 30 is present (in other words, a part where the three tubes 10, 20, and 30 are disposed side-by-side) is also referred to as a “second predetermined position”. Furthermore, an arbitrary position further toward the proximal end side of the second predetermined position (in other words, a part where the two tubes 10 and 20 are disposed side-by-side) is also referred to as a “first predetermined position” or simply a “predetermined position”.

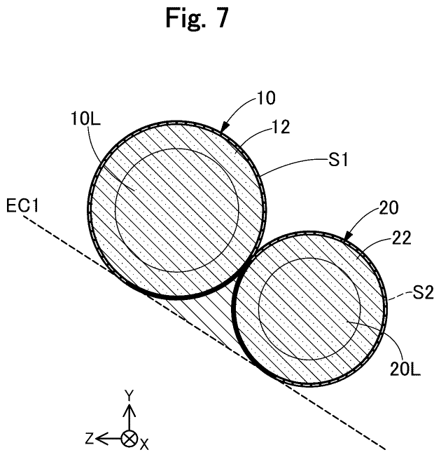

FIG. 7 is a diagram describing a first area S1 and a second area S2. FIG. 7 shows a transverse cross-section of the catheter 1 along line F-F in FIG. 1. Here, an area of a region enclosed by the outer periphery of the catheter 1 is referred to as the “first area S1”. In FIG. 7, the first area S1 is shown enclosed by a thick border and with a dot hatching. As shown in FIG. 7, the first area S1 in the transverse cross-section of the catheter 1 along line F-F is the area of a region enclosed by the outer periphery of the sensor tube 10 and the OTW tube 20.

Moreover, an area of a region enclosed by the first common external tangent line EC1 and a contour line formed by the outer periphery of the sensor tube 10 and the OTW tube 20 is referred to as the “second area S2”. In FIG. 7, the second area S2 is shown enclosed by a dashed line and with a diagonal hatching. As shown in FIG. 7, the second area S2 in the transverse cross-section of the catheter 1 along line F-F is an area of a region enclosed by the first common external tangent line EC1 and a contour line formed by the outer periphery of the sensor tube 10 and the OTW tube 20, the contour line extending toward the outer side from the first common external tangent line EC1 (indicated by a white dashed line). In other words, the contour line refers to the portions of the outer periphery of the first and second tubes that are not bounded by the common external tangent line.

As is clear from FIG. 7, the first area S1 (thick line border, dot hatching) is less than or equal to the second area S2 (dashed line border, diagonal hatching). The first area S1 is smaller than the second area S2.

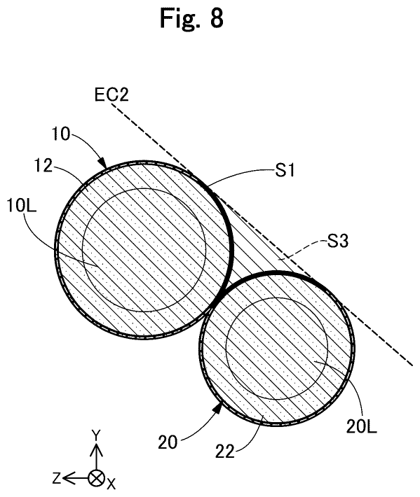

FIG. 8 is a diagram describing a first area S1 and a third area S3. FIG. 8 shows a transverse cross-section of the catheter 1 along line F-F in FIG. 1. Here, in FIG. 8, in a similar manner to FIG. 7, the area of the region surrounded by the outer periphery of the catheter 1 (the first area S1) is shown enclosed by a thick border and with a dot hatching.

Moreover, an area of a region enclosed by the second common external tangent line EC2 and a contour line formed by the outer periphery of the sensor tube 10 and the OTW tube 20 is referred to as the “third area S3”. In FIG. 8, the third area S3 is shown enclosed by a dashed line and with a diagonal hatching. As shown in FIG. 8, the third area S3 in the transverse cross-section of the catheter 1 along line F-F is an area of a region enclosed by the second common external tangent line EC2 and a contour line formed by the outer periphery of the sensor tube 10 and the OTW tube 20, the contour line extending toward the outer side from the second common external tangent line EC2 (indicated by a white dashed line).

As is clear from FIG. 8, the first area S1 (thick line border, dot hatching) is less than or equal to the third area S3 (dashed line border, diagonal hatching). The first area S1 is smaller than the third area S3.

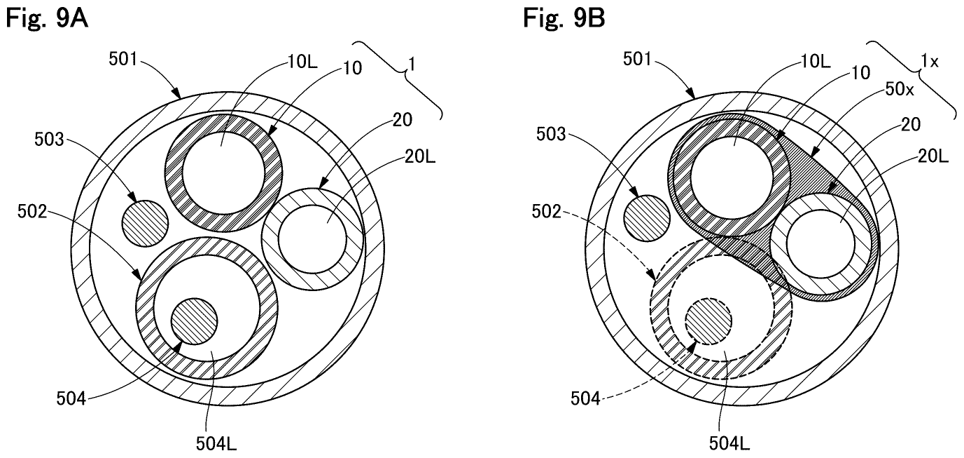

FIGS. 9A and 9B are diagrams describing the insertion of a guiding catheter. FIG. 9A shows a state where the catheter 1 of the present embodiment has been inserted into a guiding catheter 501. FIG. 9B shows a state where a catheter 1x of a comparative example has been inserted into the guiding catheter 501. FIGS. 9A and 9B show a transverse cross-section at a part where only the sensor tube 10 and the OTW tube 20 are side-by-side (line F-F), which accounts for the longest portion of the entire length of the catheter 1 and the catheter 1x.

As shown in FIG. 9A, in the catheter 1 of the present embodiment, when the catheter 1 is inserted into the guiding catheter 501, a space into which auxiliary devices (in the illustrated example, a microcatheter 502 and guidewires 503 and 504) are inserted can be secured inside the lumen of the guiding catheter 501. In other words, the catheter 1 of the present embodiment does not obstruct the insertion of auxiliary devices into the lumen of the guiding catheter 501. This is due to the catheter 1 satisfying a relationship in which the first area S1 is less than or equal to the second area S2. In other words, in the catheter 1, because the catheter 1 of the present embodiment has the gap SP (a recess or concave portion) between the first common external tangent line EC1 and the outer periphery of the catheter 1, and the gap SP (a recess of concave portion) between the second common external tangent line EC2 and the outer periphery of the catheter 1, auxiliary devices (particularly large-diameter devices such as the microcatheter 502) can be positioned in the gap SP parts of the catheter 1 as illustrated, and the space inside the lumen of the guiding catheter 501 can be effectively utilized.

On the other hand, as shown in FIG. 9B, in the catheter 1x of the comparative example, when the catheter 1x is inserted into the guiding catheter 501, the covering portion 50x acts as an obstruction, and the microcatheter 502 cannot be inserted into the lumen of the guiding catheter 501. That is, in the catheter 1x of the comparative example, there are significant restrictions on the size and number of auxiliary devices that can be inserted into the lumen of the guiding catheter 501, and the space inside the lumen of the guiding catheter 501 cannot be effectively utilized.

As described above, the catheter 1 (medical device) according to the first embodiment includes the sensor tube 10 (first tube) that constitutes the sensor lumen 10L (first lumen), and the OTW tube 20 (second tube) that constitutes the OTW lumen 20L (second lumen), and because the outer peripheral surface of the sensor tube 10 and the outer peripheral surface of the OTW tube 20 are joined, the pushing force applied by the operator can be efficiently transmitted to the distal end side. Furthermore, in a transverse cross-section at a predetermined position in the longitudinal direction of the catheter 1, the first area S1, which is the area of a region enclosed by the outer periphery of the catheter 1, is less than or equal to the second area S2, which is the area of a region enclosed by the common external tangent line EC1 of the sensor tube 10 and the OTW tube 20, and the contour line formed by the outer periphery of the sensor tube 10 and the OTW tube 20. For this reason, in the transverse cross-section of the catheter 1, although the outer diameter Lmax (maximum outer diameter) in the longitudinal direction is the outer diameter of two tubes, the outer diameter Lmin (minimum outer diameter) in the short axis direction can be made the outer diameter of one tube. Also, the catheter 1 can also have a structure in the transverse cross-section having recesses (concave portions) in parts adjacent to each of the sensor tube 10 and the OTW tube 20. As a result, according to the catheter 1 of the first embodiment, when the transverse cross-section of the catheter 1 is compared with a conventional configuration in which the transverse cross-section has a circular shape or elliptical shape that encloses the sensor tube 10 and the OTW tube 20 (for example, the configuration in FIGS. 9A and 9B), as described in FIG. 9A, the insertion of the auxiliary devices 502, 503 and 504 into the lumen of the guiding catheter 501 is less likely to be obstructed. Therefore, according to the catheter 1 of the first embodiment, in a medical device having two or more lumens, it is possible to use many auxiliary devices 502, 503, and 504 inside the limited lumen of a guiding catheter 501, and to broaden the restriction width of the auxiliary devices.

In addition, according to the catheter 1 (medical device) of the first embodiment, in the transverse cross-section of the catheter 1, because the gaps SP are formed between the common external tangent lines EC1 and EC2 and the outer periphery of the catheter 1, the auxiliary devices 502, 503, and 504 can be positioned in the gap SP parts when the catheter 1 is inserted into the guiding catheter 501. Also, according to the catheter 1 (medical device) of the first embodiment, as shown in FIG. 6A, the gap SP formed between the single common external tangent line EC1 and the outer periphery of the sensor tube 10 and the OTW tube 20 (first and second tubes) enables the catheter 1 to be made flexible even when the RX tube 30 (third tube) is present.

Further, in the catheter 1 (medical device) of the first embodiment, in a transverse cross-section at the predetermined position in the longitudinal direction, the first area S1 is less than or equal to the third area S3, which is the area of a region enclosed by the second common external tangent line EC2 of the sensor tube 10 and the OTW tube 20, which is different from the first common external tangent line EC1, and the contour line formed by the outer periphery of the sensor tube 10 and the OTW tube 20. Therefore, the catheter 1 can also have a structure in the transverse cross-section having recesses (concave portions) on both sides of parts adjacent to each of the sensor tube 10 and the OTW tube 20. As a result, according to the catheter 1 of the first embodiment, in a medical device having two or more lumens, it is possible to use even more auxiliary devices 502, 503, and 504 inside the limited lumen of a guiding catheter 501, and to further broaden the restriction width of the auxiliary devices.

Furthermore, according to the catheter 1 (medical device) of the first embodiment, because the sensor lumen 10L is a sensor lumen 10L that acquires image information, a procedure can be performed using the catheter 1 under the guidance of the sensor 70, which acquires image information of biological tissue using ultrasound waves.

Also, because the catheter 1 (medical device) of the first embodiment further includes the RX tube 30 (third tube) that constitutes the RX lumen 30L (third lumen), the pushing force applied by the hand of the operator can be even more efficiently transmitted to the distal end side. Moreover, by using the RX lumen 30L, a device such as the workhorse wire 200 can also be used together.

### Second Embodiment

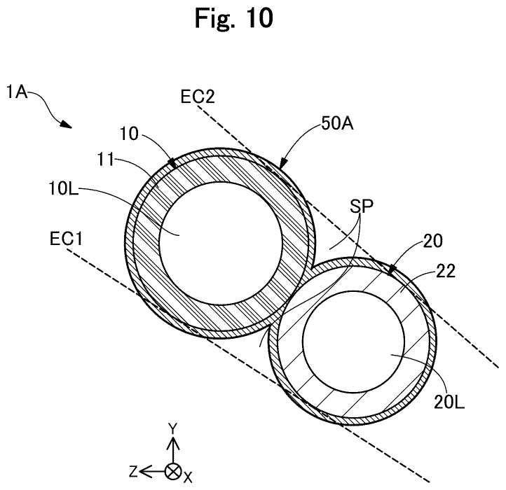

FIG. 10 is a transverse cross-sectional view of a catheter 1A according to a second embodiment. FIG. 10 shows a transverse cross-section of the catheter 1A along line F-F in FIG. 1. The catheter 1A of the second embodiment has a covering portion 50A instead of the covering portion 50 in the configuration described in the first embodiment.

The covering portion 50A is provided not only in the section described in the first embodiment (the section where the three tubes 10, 20, and 30 extend side-by-side), but also in the section where only the sensor tube 10 and the OTW tube 20 are side-by-side. In other words, the covering portion 50A is also provided in the section between the proximal end opening 302 of the catheter 1 and the first reinforcing member 61. In the catheter 1A of the second embodiment, in a similar manner to the first embodiment, the relationship in which the first area S1 is less than or equal to the second area S2, and the relationship in which the first area S1 is less than or equal to the third area S3, are established. Also, in the catheter 1A of the second embodiment, in a similar manner to the first embodiment, a gap SP is formed between the first common external tangent line EC1 and the outer periphery of the catheter 1A, and a gap SP is formed between the second common external tangent line EC2 and the outer periphery of the catheter 1A.

In this way, various modifications can be made to the configuration of the catheter 1A, and the covering portion 50A may be provided in an arbitrary section of the catheter 1A. With the catheter 1A of the second embodiment as described above, the same effects as in the first embodiment can be achieved.

### Third Embodiment

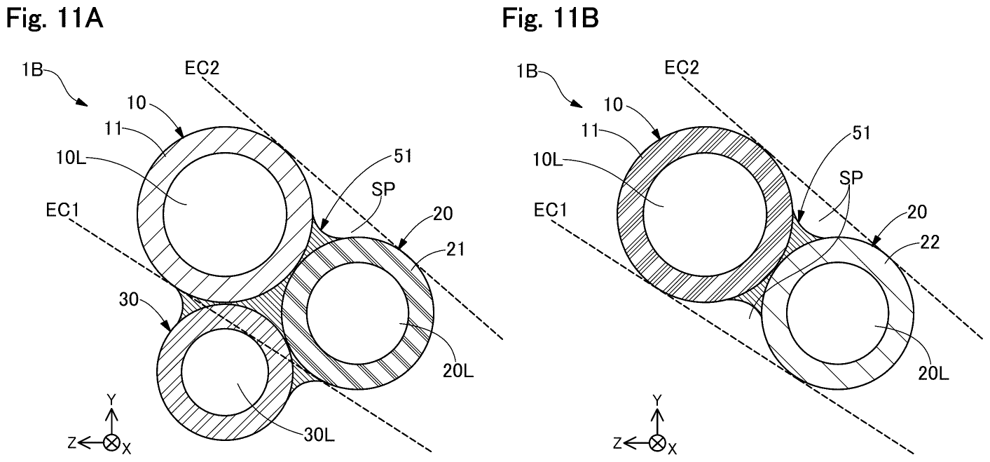

FIGS. 11A and 11B are transverse cross-sectional views of a catheter 1B according to a third embodiment. FIG. 11A shows a transverse cross-section of the catheter 1B along line D-D in FIG. 1. FIG. 11B shows a transverse cross-section of the catheter 1B along line F-F in FIG. 1. The catheter 1B of the third embodiment has a bonding agent 51 instead of the covering portion 50 in the configuration described in the first embodiment.

The bonding agent 51 is disposed in parts adjacent to the sensor tube 10, the OTW tube 20, and the RX tube 30, and joins the three tubes 10, 20, and 30 to each other. As the bonding agent 51, for example, any bonding agent such as an epoxy-based adhesive can be used. In the catheter 1B of the third embodiment, in a similar manner to the first embodiment, the relationship in which the first area S1 is less than or equal to the second area S2, and the relationship in which the first area S1 is less than or equal to the third area S3, are established. Also, in the catheter 1B of the third embodiment, in a similar manner to the first embodiment, a gap SP is formed between the first common external tangent line EC1 and the outer periphery of the catheter 1B, and a gap SP is formed between the second common external tangent line EC2 and the outer periphery of the catheter 1B.

Thus, various modifications can be made to the configuration of the catheter 1B, and the sensor tube 10, the OTW tube 20, and the RX tube 30 may be fixed by the bonding agent 51 instead of the covering portion 50. With the catheter 1B of the third embodiment as described above, the same effects as in the first embodiment can be achieved.

### Modifications of Embodiments

The present disclosure is not limited to the embodiments described above, and can be implemented in various modes without departing from the spirit thereof, and for example, the following modifications are also possible.

### [Modification 1]

In the first to third embodiments described above, an example of the configuration of the catheters 1, 1A, and 1B has been described. However, various modifications can be made to the configuration of the catheters 1, 1A, and 1B.

For example, the outer peripheral surface of the covering portion 50, or the outer peripheral surface of the catheter 1 including the covering portion 50, may be coated with a hydrophilic resin or a hydrophobic resin. For example, the sensor 70 was built into the sensor lumen 10L of the sensor tube 10, and was configured so as to not be detachable from the catheter 1. However, the sensor 70 may be configured so as to be detachable from the catheter 1. That is, the catheter 1 does not have to include the sensor 70 as a component.

For example, at least one of the distal end tip 40, the first marker 41, and the second marker 42 may be omitted. For example, the shapes of the distal end tip 40, the first marker 41, and the second marker 42 can be arbitrarily changed. The distal end tip 40 may have a constant outer diameter from the distal end toward the proximal end, and the shape in the transverse cross-section may be a non-circular symmetric shape. The first marker 41 and the second marker 42 may have a shape that is different from an annular shape (for example, a shape obtained by cutting an annular shape at an arbitrary angle, a linear shape, or a coil shape in which a wire is spirally wound).

For example, the arrangement of the distal end tip 40, the first marker 41, and the second marker 42 can be arbitrarily changed. The first marker 41 may be disposed at a position that is different from a position adjacent to the proximal end of the distal end tip 40 (such as a position that is separated from the distal end tip 40). The second marker 42 may be disposed at a position that is different from a position adjacent to the distal end of the distal end opening 201 of the OTW tube 20 (such as a position that is separated from the distal end opening 201). The first marker 41 and the second marker 42 may be disposed on a tube different from the RX tube 30 (the sensor tube 10 or the OTW tube 20). The first marker 41 and the second marker 42 may be disposed on the same tube as described above, or may be disposed on different tubes.

For example, the sensor tube 10 may be constituted by one tube, or may be constituted by three or more tubes. Similarly, the OTW tube 20 may be constituted by one tube, or may be constituted by three or more tubes. Similarly, the RX tube 30 may be constituted by two or more tubes.

For example, the shapes of the branched connector 60, the first reinforcing member 61 to third reinforcing member 63, the cylindrical member 64, the connector 65, and the connector 25 described above are merely examples, and may be arbitrarily changed. For example, at least a part of the branched connector 60, the first reinforcing member 61, the second reinforcing member 62, and the cylindrical member 64 may be configured as a single member, or may be omitted. For example, the third reinforcing member 63 and the connector 25 may be configured as a single member. For example, the cylindrical member 64 may be provided with a mechanism that assists the adjustment of at least one of the front-rear position of the sensor 70 and the orientation of the sensor 70 in the circumferential direction (such as a scales or stoppers provided at predetermined lengths in the longitudinal direction, or scales or stoppers provided at predetermined angles in the circumferential direction).

### [Modification 2]

The configurations of the catheters 1, 1A, and 1B of the first to third embodiments and the configuration of the catheters 1, 1A, and 1B of Modification 1 described above may be combined as appropriate.

### Aspects

(1) According to an aspect of the present disclosure, a medical device is provided. The medical device includes: a first tube having a first lumen; a second tube having a second lumen; and a third tube having a third lumen; wherein an outer peripheral surface of the first tube and an outer peripheral surface of the second tube are joined, and in a transverse cross-section at a predetermined position in a longitudinal direction of the medical device, a first area, being an area of a region that is enclosed by an outer periphery of the medical device, is less than or equal to a second area, being an area of a region that is enclosed by both a single common external tangent line of the first tube and the second tube, and a contour line formed by an outer periphery of the first tube and the second tube.

According to such a configuration, the cross-sectional area of the medical device at the predetermined position can be reduced. Therefore, in a medical device having two or more lumens, many auxiliary devices can be used inside the limited lumen of a guiding catheter.

(2) In the medical device according to the above aspect, in the transverse cross-section, a gap may be formed between the common external tangent line and the outer periphery of the first and second tubes. According to such a configuration, when the medical device is inserted into a guiding catheter, an auxiliary device can be positioned in the gap part.

(3) In the medical device according to the above aspect, the predetermined position may be a first predetermined position, in a transverse cross-section at a second predetermined position in the longitudinal direction of the medical device, being a transverse cross-section in which the third tube is present, a gap may formed between the single common external tangent line and the outer periphery of the first and second tubes, and the gap may be an inner cavity of the medical device. According to such a configuration, the gap formed between the single common external tangent line and the outer periphery of the first and second tubes enables the medical device to be made flexible even when the third tube is present.

(4) In the medical device according to the above aspect, in the transverse cross-section, when the common external tangent line is a first common external tangent line, the first area may be less than or equal to a third area, being an area of a region that is enclosed by both a second common external tangent line of the first tube and the second tube, the second common external tangent line being different from the first common external tangent line, and a contour line formed by the outer periphery of the first tube and the second tube. According to such a configuration, the medical device can have a structure in the transverse cross-section having recesses (concave portions) on both sides of parts adjacent to each of the first tube and the second tube. As a result, according to the present configuration, in a medical device having two or more lumens, it is possible to use even more auxiliary devices inside the limited lumen of a guiding catheter, and to further broaden the restriction width of the auxiliary devices.

(5) In the medical device according to the above aspect, the first lumen may be a lumen for a sensor that acquires image information. According to such a configuration, a procedure can be performed under the guidance of the sensor that acquires image information of biological tissue using ultrasound waves.

(6) In the medical device according to the above aspect, the first tube, the second tube, and the third tube may have respective outer peripheral surfaces joined to each other. According to such a configuration, a pushing force applied by an operator can be more efficiently transmitted to a distal end side.

Although the present aspect has been described above based on the embodiments and modifications, the embodiments of the aspect described above are for facilitating the understanding of the present aspect, and do not limit the present aspect. The present aspect can be modified or improved without departing from the spirit thereof and the scope of claims, and the present aspect also includes equivalents thereof. Furthermore, the technical features of the present aspects, if not indicated as essential herein, may be deleted as appropriate.

What is claimed is:

1. A medical device comprising:
a first tube having a first lumen; and
a second tube having a second lumen; wherein
an outer peripheral surface of the first tube and an outer peripheral surface of the second tube are joined, and
in a transverse cross-section at a predetermined position in a longitudinal direction of the medical device, a first area, being an area of a region that is enclosed by an outer periphery of the medical device is less than or equal to a second area, being an area of a region that is enclosed by both a single common external tangent line of the first tube and the second tube, and a contour line formed by an outer periphery of the first tube and the second tube.

2. The medical device according to claim 1, wherein
in the transverse cross-section, a gap is formed between the single common external tangent line and the outer periphery of the first and second tubes.

3. The medical device according to claim 1, further comprising a third tube having a third lumen.

4. The medical device according to claim 3, wherein outer peripheral surfaces of the first tube, the second tube, and the third tube are joined to each other.

5. The medical device according to claim 3, wherein
the predetermined position is a first predetermined position located in a proximal portion of the medical device, and
in a transverse cross-section at a second predetermined position located distal to the first predetermined position, in which the first tube, the second tube, and the third tube are present, a gap formed between the single common external tangent line and the outer periphery of the first and second tubes defines an inner cavity of the medical device.

6. The medical device according to claim 1, wherein
the single common external tangent line is a first common external tangent line, and
the first area is less than or equal to a third area, being an area of a region that is enclosed by both a second common external tangent line of the first tube and the second tube and a contour line formed by the outer periphery of the first tube and the second tube, the second common external tangent line being different from the first common external tangent line.

7. The medical device according to claim 1, wherein
the first lumen is configured to receive a sensor that acquires image information.

8. The medical device according to claim 1, wherein an outer diameter of the first tube is greater than an outer diameter of the second tube.

9. The medical device according to claim 1, further comprising a cover extending over at least a portion of an outer surface of the joined first and second tube.

10. A medical system, comprising:
a guiding catheter having an inner lumen; and
a medical device disposed within the inner lumen of the guiding catheter, the medical device including:
a first tube having a first lumen; and
a second tube having a second lumen, wherein an outer peripheral surface of the first tube and an outer peripheral surface of the second tube are joined to form a recess therebetween,
wherein the recess forms a channel between an outer surface of the medical device and an inner wall of the guiding catheter.

11. The medical system according to claim 10, wherein the channel is configured to receive an auxiliary medical device.

12. A method of performing a medical procedure, comprising:
inserting a guiding catheter having an inner lumen into a biological lumen;
advancing a medical device through the inner lumen of the guiding catheter, the medical device including a first tube and a second tube having their outer peripheral surfaces joined, wherein a cross-sectional profile of the medical device defines at least one recess; and
advancing an auxiliary device through the inner lumen of the guiding catheter, wherein the auxiliary device is positioned at least partially within the recess of the medical device.
